(19) United States
(12) Patent Application Publication
(10) Pub. No.: US 2008/0175889 A1
(43) Pub. Date: Jul. 24, 2008

(54) METHOD AND COMPOSITION FOR COATING WOUNDS AND PROTECTING SKIN USING A POLYETHER POLYURETHANE SOLUTION

(76) Inventor: Daniel Perlman, Arlington, MA (US)

Correspondence Address: WESLEY B. AMES, 7031 LOS VIENTOS SERENOS, ESCONDIDO, CA, 92029, US

(21) Appl. No.: 11/626,373

(22) Filed: Jan. 23, 2007

(51) Int. Cl. A61K 9/70; A61F 13/02; A61K 36/00

(52) U.S. Cl. 424/448; 424/195.18; 424/443; 424/447

(57) ABSTRACT

A solution of polyether polyurethane resin dissolved in a solvent system containing 2-methyloxolane (2-MeOX) solvent and at least one volatile co-solvent. The solution may also contain a functional amount of solubilized adhesive agent. Upon drying, the resulting protective elastic film has good adhesion to human and other animal skin. The solution penetrates vital tissue to a lesser extent than a comparable solution prepared using tetrahydrofuran solvent.

### RELATED APPLICATIONS

NOT APPLICABLE.

### FIELD OF THE INVENTION

The present invention relates to methods and compositions for protecting intact skin from physical insult, reddening, and/or blister formation, as well as for protecting damaged skin, e.g., wounds, abrasions, cracking, and the like on human or other animal skin using a polymeric solution that can be applied to the skin by dipping, spraying, or brushing, for example, and that dries onto the skin to form a water-resistant protective coating.

### BACKGROUND OF THE INVENTION

The following discussion is provided solely to assist the understanding of the reader, and does not constitute an admission that any of the information discussed or references cited constitute prior art to the present invention.

Protecting wounds, skin abrasions, blisters and the like on human and animal skin from further physical damage and/or contamination by the environment including wind, dirt, water, insect bites, etc. has been difficult and challenging. Most treatments that involve applying coatings to the skin are ineffective and/or require frequent replacement. Coatings placed over compromised skin may even aggravate the original injury. U.S. Pat. Nos. 5,192,536, 5,688,498, 5,942,239, and 6,030,633 disclose useful methods and compositions for coating a wound with a polyether polyurethane copolymer (PEPU) resin. Each of those references is incorporated hereby by reference in its entirety, and the references are applicable to the present invention for their descriptions of proportions of solution components, adhesive agents, germicides, and uses of film-forming solutions as skin coatings.

Huprich, in U.S. Pat. No. 5,192,536 describes a method for coating a wound in which PEPU is dissolved in tetrahydrofuran (herein abbreviated THF).

Huprich et al., in U.S. Pat. No. 5,688,498 describes a similar solution of PEPU dissolved in THF that is improved by the addition of benzoin gum as an adhesive agent.

Huprich et al., U.S. Pat. No. 5,942,239 describes solutions similar to those above but that include a fast drying solvent selected from cyclohexanone, methylethyl ketone, toluene, acetone, methanol, ethanol, propanol, isopropanol, propylene, and combinations thereof.

Hemling et al., in U.S. Pat. No. 6,030,633 describes a similar solution forming an elastic film on the skin, in which the carrier contains PEPU, benzoin gum and up to 15% nitrocellulose to further improve adhesion of the composition to the skin.

### SUMMARY OF THE INVENTION

The present invention provides an advantageous protective skin covering that utilizes a solvent system having improved safety characteristics with a polyether polyurethane copolymer (PEPU) resin. In particular, the solvent system used for the solutions for the present skin coverings exhibit less penetration following topical administration than otherwise equivalent solutions that utilize tetrahydrofuran (THF) as the solvent for the PEPU resin.

Thus, a first aspect of the invention concerns a solution that includes a polyether polyurethane resin dissolved in 2-methyloxolane (2-MeOX) solvent and preferably including at least one co-solvent, where the solution dries to a protective elastic film having good adhesion to human and other animal skin.

In particular embodiments, the solution includes 3-30%, 5-25%, 5-20%, 5-15%, 5-12%, or 5-10% by weight of a polyether polyurethane resin. In particular embodiments, the solution also contains a functional amount of a solubilized adhesive agent, e.g., benzoin gum, and/or a plasticizer; benzoin gum is present as an adhesive agent at a concentration of 1-20%, 1-15%, 1-10%, 2-10%, 2-8%, 2-5%, 3-7%, 4-6%, or 5-10% by weight of the solution (where the ranges are inclusive of the end points); the benzoin gum and the polyether polyurethane together are 5-50%, 5-30%, 5-25%, 7-25%, 7-20%, or 10-20% by weight of the solution; the weight ratio of polyether polyurethane to benzoin gum is 0.6, 0.7, 0.8, 0.9, 1.0, 1.25, 1.5, 1.75, 2.0, 2.2, 2.5, 3.0, 3.5, or 4.0±15% for each value.

In certain embodiments, the solution includes from 5 to 20 parts by weight of polyether polyurethane resin, from 50 to 90 parts by weight of 2-methyloxolane solvent, and from 5 to 40 parts by weight of an organic co-solvent, and in particular embodiments includes from 2 to 10 parts by weight of solubilized plasticizer and/or adhesive agent, e.g., benzoin gum adhesive (or 1-15 parts, 2-8 parts, 2-5 parts, 3-7 parts, 4-6 parts, or 5-10 parts); the weight ratio of co-solvent(s) to 2-methyloxolane is 0.05, 0.06, 0.07, 0.08, 0.09, 0.10, 0.11, 0.12, 0.14, 0.15, 0.17, 0.20, 0.25, 0.30, 0.35, 0.40, 0.45, or 0.50±10% or in a range of 0.05-0.10, 0.10-0.15, 0.12-0.15, 0.12-0.17, 0.14-0.20, 0.20-0.30, 0.30-0.50.

In particular embodiments, the solution penetrates vital tissue to a lesser extent than a comparable solution prepared using tetrahydrofuran solvent; the solution includes nitrocellulose, e.g., at a concentration of 0.1-15%, 0.2-15%, 0.5-15%, 0.5-10%, 0.5-5%, 1-10%, 1-5%, or 2-5%.

In particular embodiments, the organic co-solvent is a ketone solvent, for example, acetone and/or methyl ethyl ketone (MEK), e.g., in a ratio with 2-MeOX as indicated above.

In certain embodiments, the solution also includes functional amounts of one or more additional components, such as antiseptic agent or germicide, humectant, emollient, and/or colorant; an antiseptic agent or germicide is present as 0.01-2.0%, 0.02-1.5%, 0.02-1.0%, 0.05-1.5%, 0.5-1.0% by weight of the solution; the antiseptic agent or germicide is benzalkonium chloride, benzethonium chloride, linear or branched chain fatty acids, sodium pyradinathione, polyhexamethylene biguanide, chlorhexidine diacetate, or quaternary ammonium compounds or combinations thereof.

In a related aspect, the invention provides a fluid skin covering formed by applying a solution comprising polyether polyurethane (PEPU) resin dissolved in 2-methyloxolane (2-MeOX) solvent and including at least one co-solvent applied on the skin of a human or other animal, wherein said solution dries to a protective elastic film having good adhesion to human and other animal skin (e.g., a solution according to the preceding aspect or an embodiment thereof).

Yet another related aspect concerns an elastic skin covering that is or includes a protective elastic film on the skin of a human or other animal resulting from drying of a solution comprising polyether polyurethane (PEPU) resin dissolved in 2-methyloxolane (2-MeOX) solvent and including at least one co-solvent (e.g., resulting from drying of a solution as described for the first aspect above or an embodiment thereof). Preferably the film has good adhesion to the skin.

The solutions and resulting elastic films can be applied to a variety of different animals and is especially for protective coatings on particular anatomical parts, e.g., one or more of humans, bovines (such as cows, and in particular on their udders, including the teats), equines, ovines, caprines, porcines, felines, canines, and avians.

Advantageously, the present solutions can be provided in kit form. Thus, another related aspect concerns a skin protection kit that includes a selected volume of a solution (e.g., in a liquid-tight container) that includes polyether polyurethane (PEPU) resin dissolved in 2-methyloxolane (2-MeOX) solvent and at least one co-solvent, where the solution dries to a protective elastic film having good adhesion to human and other animal skin. The kit also includes an applicator suitable for applying the solution to the skin and/or instructions for using the solution.

In particular embodiments, the solution is as described for one or more of the preceding aspects or embodiments.

In certain embodiments, the volume of solution is 10-100 ml, 50-100 ml, 100-500 ml, 100-200 ml, 500-1000 ml, or greater than 1000 ml; the applicator includes a brush, a spray device (e.g., a pump spray or a pressurized spray can, or an absorbent material (e.g., a sponge, swab, or absorbent pad).

The amount (concentration) of adhesive agent in the solution can be varied, e.g., to adapt to different animal skins, application procedures, and/or temperatures. Thus, another aspect of the invention concerns a process for selecting a skin-covering solution for a particular application or set of animal skin-covering applications, by varying the type and/or amount of adhesive agent in a skin-covering solution to optimize adhesion of the film formed by the solution on the skin in the particular application, where the solution includes polyether polyurethane (PEPU) resin dissolved in a mixed solvent comprising 2-methyloxolane (2-MeOX) solvent and at least one co-solvent, and a functional amount of the adhesive agent. A solution with a particular type and/or concentration of adhesive agent is selected having desirable or optimized properties. In particular embodiments, the solution is as described for an aspect or embodiment above.

In certain embodiments, the desirable or optimized property is or includes adhesion strength, adhesion lifetime, or peelability.

Also in particular embodiments, the application concerns application of the solution on the skin of one or more (e.g., in any combination of 2, 3, 4, 5, or more) of humans, bovines (such as cows), equines, ovines, caprines, porcines, felines, canines, and/or avians.

Likewise, still another aspect concerns a process for protecting human and other animal skins by applying a solution as described for an aspect above to the animal's skin.

In particular embodiments of this aspect, the solution is as specified for an embodiment of an above aspect; the animal is a human, bovine (such as a cow), equine, ovine, caprine, porcine, feline, canine, or avian.

In certain embodiments, the solution is applied to the skin using a method selected from the group consisting of dipping, spraying, spreading, brushing and wiping.

A further aspect of the invention concerns a method for making a film-forming solution (e.g., a solution as described above), by solubilizing or dispersing polyether polyurethane resin in 2-MeOX solvent; preferably the solvent also includes an organic co-solvent as described herein. The method can also include solubilizing or dispersing an adhesive agent such as benzoin gum in the solvent, before and/or after dissolving the polyether polyurethane. The method can also include solubilizing or dispersing one or more additional functional agents in the solvent, such as an antiseptic agent or germicide, a colorant or dye, a humectant, or an emollient.

In addition to the use of the use of the 2-MeOX/co-solvent mixed solvent system with a polyether polyurethane resin, the invention also concerns the use of this solvent system in any other skin contact application with components soluble in this solvent system. Examples can include skin softener solutions and emulsions and the like.

The invention also concerns the use of the present solvent system with other polymers for protective skin coatings. Thus, for example, the invention includes the aspects above in which the polyether polyurethane is replaced with a polyester polyurethane, as well as other polymers compatible with skin contact applications.

For terms that are not defined below, the common definition is assumed as provided in the current edition of Webster's International Dictionary or for chemical terms as provided in a standard organic chemistry textbook such as Organic Chemistry (5^{th Edition}) by Leroy Wade (Prentice-Hall, Inc.

The term “polyether polyurethane resin” has its conventional meaning in the field of polyurethane copolymer resins referring generally to copolymers of polyisocyanates (often diisocyanates) and polyols (often diols), such as polyethylene glycol, polypropylene glycol, and polytetramethylene glycol, although other components may also be included in the material. A description of many types of polyether polyurethane resins is provided in U.S. Pat. No. 6,777,524 by Shimizu, et al. as follows: “A polyether polyurethane is produced from a polyisocyanate, a high molecular weight diol and a low molecular weight diol as main raw materials, and is a block copolymer having a soft segment composed mainly of the high molecular weight diol and a hard segment composed mainly of the polyisocyanate and the low molecular weight diol. By virtue of such structure, the polyether polyurethane exhibits rubber elasticity. The chemical composition, length of the polymer blocks, and secondary and tertiary structures of the polyether polyurethane depend mainly on the types of the polyisocyanate and the high molecular weight diol used, and have a large influence on the physical properties of an ultimate polyether polyurethane product. Especially, the choice of the soft segment is a very important factor in fields where a product obtained from a polyether polyurethane, such as an elastic fiber or a polyurethane elastomer, is required to have excellent and precisely controlled mechanical properties and visco-elastic properties.”

The term “protective elastic film” refers to a polyether polyurethane polymeric film for topical application to the human (or other animal) skin, that can be dissolved in a solvent system (that may include a co-solvent) compatible with forming a coherent evaporative coating that is preferably strong, elastic, adherent, water-resistant and breathable when dried onto the skin.

The term “evaporative liquid composition” or “evaporative coating” refers to a liquid coating in which non-volatile components dissolved or dispersed in the liquid, form a flexible film by simple evaporation of the solvents present in the liquid after the liquid has been applied to the skin.

The term “solvent” or “volatile solvent”, as used in the context of the present invention, means a liquid chemical, such as 2-methyloxolane, that is capable of dissolving a coating material, e.g., a polyether polyurethane resin alone or with a co-solvent, and maintaining the solubility of the coating material at room temperature. The solvent should evaporate with sufficient rapidity when coated onto human or animal skin so that most of the liquid, preferably 80% or more, will evaporate within approximately five minutes and preferably within 2 minutes or less.

In the context of the present invention, the term “co-solvent” refers to one or more organic molecular species that are liquid and volatile at room temperature such as the ketones (e.g., acetone and/or methylethyl ketone) that can aid in dissolving and/or maintaining solubility of a polymeric material such as PEPU when used in combination with certain solvents (e.g., 2-MeOX). Co-solvents are preferably sufficiently volatile so as to evaporate at a rate comparable to, or even faster than the primary solvent(s). In this fashion, the co-solvent does not retard the drying or immobilization of a polymeric coating on the skin.

The terms “antiseptic” and “germicide” as used herein refer to a substance or agent suitable for topical use (e.g., such as benzalkonium chloride or benzethonium chloride approved by the FDA) that prevents or arrests the growth of microorganisms (e.g., bacteria, viruses and molds) in a skin wound, burn, or other skin injury.

As used in connection with the present solutions, the term “adhesive agent” refers to a compound or mixture of compounds that increases the adhesion to skin of coatings resulting from solvent evaporation of the solution on skin. Benzoin gum is one example of such an adhesive agent. Such adhesive agents include materials that are referred to as “tackifiers”.

In the present context, a “plasticizer” is any of variety of substances that may be added to the liquid coatings of the present invention to render the resulting dried PEPU resin coating more pliable, conformable, and/or extensible. For example, the 2000 and 5000 series of Escorex™ aromatic modified aliphatic, aliphatic, and cycloaliphatic tackifying resins (ExxonMobil Chemical Company, Houston, Tex.), as well as the aromatic hydrocarbon resins of the Resinall Corporation (Stamford, Conn.) are suitable alternatives or additional agents for benzoin gum as tackifiers and plasticizers.

In the context of the solutions of the present invention, the term “functional amount” refers to an amount of a solution component that is sufficient to provide an objectively useful level of the indicated activity in a skin coating application, e.g., a useful level of germicidal activity for a germicide, or a useful level of coloration for a dye or colorant. This is distinguished from a de minimis level of activity.

Additional embodiments will be apparent from the Detailed Description and from the claims.

### DETAILED DESCRIPTION OF THE PREFERRED EMBODIMENTS

As indicated in the Summary above, the present invention concerns advantageous skin covering solutions. These solutions incorporate a solvent system that generally produces less skin penetration and tissue absorption and/or less skin irritation than the previously used tetrahydrofuran (THF), while at the same time providing effective functional solvent characteristics for dissolution of the film-forming polymer and formation of the elastic film on the skin when applied.

Thus, the present preparations include a polyether polyurethane (abbreviated PEPU) resin polymer and additional optional soluble ingredients such as benzoin gum (an adhesive agent), antiseptic agents or germicides, humectants, emollients, colorants, and the like are dissolved in 2-methyloxolane, CAS Registry Number 96-47-9 (herein abbreviated 2-MeOX). (2-MeOX is also known as 2-methyltetrahydrofuran and therefore can be regarded as a derivative of THF). Preferably the 2-MeOX is used together with at least one co-solvent with desirable properties to form a mixed solvent system. Thus, one (or more) co-solvents that are polar organic solvents, e.g., ketones such as acetone and/or methylethyl ketone (MEK) may be added to the 2-MeOX to enhance the solubility of the PEPU resin in 2-MeOX.

The relative proportions of the 2-MeOX and the co-solvent(s) can be varied to provide desired properties, such as evaporation rates and/or ability to solubilize the PEPU resins. In most cases, for the solvent system from approximately 2% to 50% by weight of co-solvent(s) may be combined with 2-MeOX. For example, 10% by weight of acetone combined with 90% by weight 2-MeOX is a useful solvent-co-solvent blend for dissolving and maintaining the solubility of PEPU resins.

The 2-MeOX with optional co-solvents provides a PEPU polymer-based solution that can be safely and effectively applied to animal and/or human skin. Applicant has discovered that during storage of the dissolved PEPU resin, the presence of a co-solvent helps prevent some or most of the PEPU material from slowly and undesirably separating or precipitating as a gel phase in the liquid. The polymer solution coating dries rapidly to form an elastic PEPU film that is vapor permeable, waterproof, wind-proof, dirt-proof, insect-proof, etc. This polymer-containing solution provides distinct advantages over the film-producing solutions described in U.S. Pat. Nos. 5,688,498 and 6,030,633. As discussed below, prior PEPU polymer-containing solutions were based on the use of the industrial solvent known as tetrahydrofuran (THF, CAS Registry Number 109-99-9).

In view of the biological issues associated with the use of THF as a PEPU resin solvent, Applicant searched for a solvent that would have better toxicological properties than THF, while at the same time possessing: (i) PEPU polymer-solubilizing properties similar to THF, (ii) a sufficiently low boiling point and therefore adequate vapor pressure to allow rapid air drying of PEPU polymer films (when applied to the skin), (iii) low acute toxicity and a reduced tendency to cause skin irritation, skin sensitization and other health problem associated with skin contact and breathing of THF solvent vapor as the urethane coating dries on the skin.

There are few suitable rapidly evaporating solvents of low toxicity, that are non-irritating and non-sensitizing to the skin, and that can also be used for dissolving regular polyester-polyurethane resins. There are even fewer solvents that meet the above criteria and that can also dissolve the more challenging polyether-containing PEPU resin. For example, while some polyester-polyurethane resins may be soluble in low toxicity easy-to-use solvents such as MEK and acetone, the polyether-containing polyurethane resins (PEPU resins) of the present invention will not dissolve in these common volatile solvents. While cyclohexanone and dimethylformamide are known to dissolve most polyurethanes including PEPU resins, both are toxic, have high boiling points (156° C. and 153° C. respectively) and are not sufficiently volatile to be useful as the primary solvent in a product that must dry rapidly on the skin. Such slow-to-evaporate solvents would allow a polyurethane-based coating to remain tacky for a prolonged period of time. The resulting tacky coating would be inconvenient to use, attracting dirt while being compromised in its physical integrity. In practice, for an evaporative solvent to be useful in the present invention, it should preferably have a boiling point below 100° C.

As part of the investigations to develop a useful alternative solvent to THF, Applicant tested the lacquer-type solvent alternatives that are described by Hemling et al., and found that they are essentially non-functional as solvents for PEPU resins. In the case of cyclohexanone (and dimethylformamide that is not mentioned by Hemling et al.), these solvents are capable of dissolving PEPU resins, but are both toxic and slow-to-evaporate, making them undesirable for use in the present invention.

Even though 2-MeOX has primarily been used as a solvent for generating higher concentrations and improved yields of Grignard reagents, Applicant unexpectedly found that the 2-MeOX solvent might be useful in a skin coating solution with PEPU. Initially Applicant found that while 2-MeOX can dissolve PEPU, that solubilization is slow at room temperature. In fact, when PEPU resin beads are suspended in 2-MeOX, the beads are observed to swell in volume many fold without dissolving. By applying heat and/or shear forces to the solvent-swollen beads, the resin can be made to dissolve. It appears that the larger molecular structure of 2-MeOX compared to THF, the substituent methyl group on the cyclic ether ring reduces the rate of solubilizing PEPU resin.

Without being bound or limited by theory, the ability to dissolve a polymer such as a PEPU resin may depend upon appropriate bonding strengths among a set of three different component forces of attraction that a given solvent molecule can exert on a potential solute molecule such as the PEPU resin molecule. Charles Hansen (Hansen, Charles M., “The Three Dimensional Solubility Parameter—Key to Paint Component Affinities: I. Solvents Plasticizers, Polymers, and Resins,” Journal of Paint Technology, Vol. 39, No. 505, 1967; Hansen, Charles M., “The Three Dimensional Solubility Parameter—Key to Paint Component Affinities: II. Dyes, Emulsifiers, Mutual Solubility and Compatibility, and Pigments,” Journal of Paint Technology, Vol. 39, No. 511, 1967; Hansen, Charles M., “The Three Dimensional Solubility Parameter—Key to Paint Component Affinities: III. Independent Calculations of the Parameter Components,” Journal of Paint Technology, Vol. 39, No. 511, 1967) developed three so called “Hansen solubility parameters” to describe solvents and other molecules. Partly empirical and partly based on thermodynamics, the three component intermolecular bonding forces are van der Waals forces, dipole forces, and hydrogen bonding forces.

In comparing Hansen numbers for THF and 2-MeOX, Applicant noticed that the polar parameter for the 2-MeOX solvent was weaker than for THF.

Given that THF was known to be an effective solvent for PEPU, Applicant hypothesized that the solvent properties of 2-MeOX might be beneficially altered to provide a more effective PEPU solvent system by adding a small amount of a polar organic “co-solvent” such as a ketone, e.g., acetone or MEK, to 2-MeOX. That strategy, in fact, was successful. The ability of 2-MeOX, in combination with a co-solvent (and advantageously other additives), to function quite effectively in a skin coating solution was tested, confirmed and perfected, resulting in the present invention. It is interesting that THF functions very well as a solvent for PEPU resins without any co-solvent, whereas 2-MeOX has a more limited ability to solubilize PEPU resins and benefits greatly from the presence of a co-solvent. Surprisingly, MEK which differs from acetone only by containing an ethyl rather than a methyl side group, is less effective as a co-solvent compared to acetone for the purposes of helping maintain the solubility of PEPU resins.

Thus, to maintain and enhance the solubility of PEPU in 2-MeOX, a small amount of polar organic co-solvent such as the ketone, acetone, is preferably added to the 2-MeOX. For example, 5%-20% by weight acetone may be added, and the lower levels, e.g., 10%, seem to be as effective as or even more effective than higher levels which, over time, may allow some PEPU gellation to occur.

It would not be evident to polymer solution formulation chemists why the use of 2-MeOX for dissolving PEPU would provide any advantage over the prior art use of THF or that it would be an effective solvent. In fact, 2-MeOX is at least 50% more expensive than THF, and its boiling point is higher (80° C. vs. 60° C.) so that evaporative drying of a PEPU film in the new solvent is slower, requiring approximately twice as long as with THF. It would seem that THF has the advantage of being entirely water-soluble, i.e., miscible, in all proportions, while only approximately 14% by weight 2-MeOX can dissolve in water [and even less water (4%) can dissolve in 2-MeOX]. The greater water solubility of THF over 2-MeOX would therefore usually be considered an advantage for preparing and using pharmaceutical formulations.

Paradoxically however, Applicant finds the opposite to be true, and that 2-MeOX has an important chemical and biological advantage over THF. More specifically, Applicant finds that much less 2-MeOX compared to THF solvent can penetrate into an animal's tissue after applying the solvents (or solvents containing dissolved PEPU polymer) onto the skin. Accordingly, there will be a considerably reduced probability that some biologically harmful interaction between solvent and living cells will occur within the human or within an animal. Less solvent penetration translates to less biological impact, less potential irritation and less sensitization with respect to the living tissue of the animal. In fact, the exceedingly low toxicity (very high LD-50) for 2-MeOX, that has been reported for both skin contact and inhalation in the rat model, i.e., approximately 6 g/kg, confirms Applicant's hypothesis. In fact, Applicant suggests that 2-MeOX may be an excellent replacement for THF in a variety of human skin contact applications.

With regard to toxicities of these solvents, most of the published literature discusses the toxicity of THF rather than 2-MeOX. The OSHA Permissible Exposure Limit and NIOSH Recommended Exposure limit for THF are both reasonably high (200 ppm, 590 mg/m³ TWA), and the LD-50 (rats) for THF is high (890 mg/kg) indicating low acute toxicity. THF is not considered a carcinogen or a teratogen, nor is it genotoxic or particularly toxic to developing embryos. THF is described as a skin irritant with a hazard of absorption and penetration into the skin. THF is also described as irritating to eyes and respiratory organs, and at high acute levels may cause a variety of symptoms.

Another potential health hazard that Applicant has discovered, relating to the use of THF as a polymer solvent applied to the skin, stems from the ability of THF to dissolve lipids (as well as its miscibility with water). In principle, this combination of properties allows the solvent to readily penetrate the skin and cellular membrane lipid barriers, and affect internal cellular processes, while entering the bloodstream. This potential hazard is supported by the observation of Diaz et al. (Diaz et al., 1992, J Neurosci Res 31(1):136-145) who found that THF can readily dissolve all of the lipids present in total brain white matter.

In connection with consideration of biological effects and hazards of solvents, while Hemling et al., in U.S. Pat. No. 6,030,633 lists many alternative solvents that may be useful carriers for combining PEPU, nitrocellulose, and benzoin gum constituents, neither this reference, nor any other reference that Applicant has found, addresses the health and toxicity issues relating to the selection of solvents used in PEPU polymer coatings applied to the skin.

Since the present invention involves applying these solvent solutions containing PEPU to the human skin, as well as to highly sensitive and highly vascularized organs of farm animals such as the teats and udders of cows, it is especially important to minimize the penetration of solvents and their absorption into such living tissues. For repeated skin contact, and also for considering potential inhalation hazards, Applicant believes that 2-MeOX will have higher safe exposure limits than THF because it is far less soluble in the aqueous-based body fluids including the blood and lymphatic tissues.

Applicant has also found another intriguing advantage in the use of the 2-MeOX solvent rather than THF. For example, 2-MeOX can be readily produced from agricultural waste (a renewable natural resource). More specifically, it is produced by hydrogenation and methylation of furfural that is fractionated and purified from corn cobs. By contrast, THF is currently commercially produced only from petroleum, a non-renewable resource.

The PEPU film protects sensitive, chafed or injured skin from further damage during the healing process and during exposure of the skin to the environment. Adhesion to the skin is improved by the addition of benzoin gum to the liquid formulation. Application methods for placing the polymer solution on the skin may be selected, and are affected by the apparent viscosity of the medium. Apparent viscosity is affected by concentration of solids, the type of solids, and the solvents used. These can each be adjusted to meet specific needs.

It is clear from the patents cited in the Background (U.S. Pat. Nos. 5,192,536, 5,688,498, 5,942,239, and 6,030633) that the particular film-forming polymers and adhesive agents included in a polymeric coating applied to the skin tend to affect the time, i.e., duration of adherence before film peeling occurs, and the extent of skin protection provided. Indeed, adhesion may be affected by both the coating ingredients, and the method used for applying the coating to the skin.

Adhesion of the protective PEPU film produced following solvent evaporation, affects the length and completeness of protection. Apparent viscosity of the solution affects the application efficacy and amount of liquid that will remain immobilized on the skin without the liquid dripping from, or running off the skin. The amount of liquid applied to the skin, in turn affects the thickness of the dried polyurethane coating, and resultant adequacy of protection. A heavier film may help improve resistance of the film to attack by outside elements, and thereby reduce adverse effects of the environment.

Specifically, changing the proportions of solvent and PEPU can increase or alternatively decrease the viscosity of the solution and convert the formulation from a spreadable or brushable solution into a dippable solution suitable for body extensions like teats, and limbs.

Adhesion of the coating can be enhanced by using an adhesive agent and/or plasticizer in the solution. For example, according to U.S. Pat. No. 5,688,498, addition of 5% by weight benzoin gum (CAS Registry Number 9000-05-9) to a solution of polyurethane dissolved in THF containing approximately 10% by weight PEPU almost doubles the lifetime, i.e., the adhesion time, of the film coating to the skin.

The benzoin resinoid or gum is derived from a tree native to South-East Asia, Styrax benzoin, with another variety of the benzoin gum derived from at least one additional Styrax species. Its trunk exudes a gum well known for its antiseptic and anti-inflammatory action. For this reason it is used externally to fight tissue inflammation and to provide disinfection of wounds. Benzoin gum is widely used in cosmetics as an antioxidant in oils, as a fixative in perfumes and as an additive to soaps. Other gums, adhesives (including materials referred to as tackifiers), and plasticizers may be substituted for, or added to the benzoin resinoid, and are within the scope of the present invention.

Addition of other ingredients may affect the useful service life of the film as well.

With regard to these polymeric solution coatings that dry on the skin by solvent evaporation, application of such coatings to the skin may be accomplished by dipping, spraying or spreading using a device such as a wipe or brush-on device. The particular coating composition, including its viscosity and rate of drying may determine the method chosen for applying the coating to the skin. In most cases it will be preferred for the viscosity of the solution to be in the range of 30 to 5000 centipoises as determined using a Brookfield viscometer with a number 3 rotor at 22 degrees C. Commonly the viscosity will be in a range of 30-3000, 30-2000, 30-1000, 30-500, 30-300, 50-2000, 50-1000, 100-1000, 100-500, or 100-300 centipoises. Of course, the lower viscosities (e.g., viscosities or viscosity ranges under about 500 or even 300 centipoises) are usually selected for spray applications although higher viscosities can also be used with suitable spray apparatus. The viscosity can be modified by altering the concentrations of the PEPU resin and/or the adhesive agent, and/or by adding viscosity enhancing agents, which may be thixotropic agents. When the resulting solution is a thixotropic solution, viscosity determinations should be carried out under conditions suitable for determining viscosity of non-Newtonian fluids and the cited viscosity should be the viscosity following shear thinning.

As indicated in the Background, the patents cited in the Background (U.S. Pat. Nos. 5,192,536, 5,688,498, 5,942,239, and 6,030,633) provide information applicable to the present invention and each is incorporated herein by reference in its entirety, including, for example, for their respective disclosures of solution component proportions and selections for particular components. These include for example, the proportions of polyether polyurethane resin, adhesive agent (e.g., benzoin gum), solvent, germicide, and colorants. The proportions and selections indicated in those references can be used in the present solutions.

For example, antiseptic (i.e., germicidal) agents can be added. The antiseptic (germicide) should be physically and chemically compatible with the remainder of the solution, or at least with the polyether polyurethane resin and the solvent system, and highly preferably with benzoin gum. By physically and chemically compatible is meant that the antiseptic is chemically and physically stable at normal room temperatures and does not significantly decrease the chemical and/or physical stability of the other solution components or of the solution as a whole. Thus, a suitable germicidal agent can be selected in view of the particular solution composition, e.g., solvent system, and intended application (e.g., with acceptable effectiveness against pathogens likely to be encountered in the particular application).

### EXAMPLE

As described above and in the claims, the present skin coating compositions may be formulated in many different ways, e.g., by varying the relative proportions of the major components. Following is one general example of the present compositions.

### Example 1

A polyether polyurethane skin coating composition may be constituted from the following ingredients (parts by weight):
(a) 50-90 parts 2-methyloxolane solvent (Penn Specialty Chemicals, Memphis, Tenn.)
(b) 5 to 40 parts polar organic solvent such as acetone
(c) 5 to 20 parts polyether polyurethane (PEPU) pellets, e.g., Estane 5714 TPU (Noveon, Inc., Cleveland, Ohio)

(d) 2 to 10 parts benzoin resinoid, aka benzoin gum (Frutarom Meer Corp., North Bergen, N.J.).

These ingredients are combined, warmed to between approximately 35-50 degrees Celsius and agitated in a chemical mixing vessel to accelerate dissolution, until a homogeneous solution is obtained.

It may be convenient to pre-mix and dissolve approximately 5 parts by weight benzoin gum with 5 parts by weight 2-MeOX, and then dilute this 50% pre-mixed benzoin gum solution approximately 10-fold into the balance of ingredients, i.e., into approximately 90-120 parts by weight of the solvent plus co-solvent blend containing 10-20 parts PEPU resin that has been solubilized. The resulting ratio of benzoin gum to PEPU resin is approximately 1:2, producing a dried film that adheres well to the skin. Including less benzoin gum produces a dried film that is less sticky and less adhesive to the skin.

Depending upon the method used for applying the liquid to the skin, the solution viscosity can be adjusted by varying the proportions of solvent and solute.

A functional amount of nitrocellulose may be optionally added and dissolved in the solution as an auxiliary polymer that may be useful in the dried coating. For example, the nitrocellulose may be present at a concentration of about 0.5% to 15% by weight of the solution.

A functional amount of an antiseptic such as benzalkonium chloride may also be optionally added and dissolved in the solution to help prevent skin infections if the solution is applied over skin cuts and abrasions.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made, among others, to the proportions of solution components and to the size distribution of the polyether polyurethane molecules. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention. Further, specification of a numerical range including values greater than one includes specific description of each integer value within that range. Where ranges are specified, unless otherwise indicated, the endpoints of the range are included.

Thus, additional embodiments are within the scope of the invention and within the following claims.

What is claimed is:

1. A solution comprising
polyether polyurethane (PEPU) resin dissolved in a solvent system comprising 2-methyloxolane (2-MeOX) solvent and at least one co-solvent.

2. The solution of claim 1, wherein upon application of said solution to human or other animal skin, said solution dries to a protective elastic film adhered to said human or other animal skin.

3. The solution of claim 1, wherein, following application on animal skin, said solution penetrates vital tissue to a lesser extent than a comparable solution prepared using tetrahydrofuran solvent.

4. The solution of claim 1, wherein said solution also contains a functional amount of a solubilized adhesive agent or plasticizer or both.

5. The solution of claim 4, wherein said adhesive agent is benzoin gum.

6. The solution of claim 5, wherein said benzoin gum comprises from 2% to 10% by weight of said solution.

7. The solution of claim 1, comprising:
from 5 to 20 parts by weight of polyether polyurethane resin;
from 50 to 90 parts by weight of 2-methyloxolane solvent; and
from 5 to 40 parts by weight of an organic co-solvent.

8. The solution of claim 7, further comprising from 2 to 10 parts by weight of solubilized benzoin gum adhesive.

9. The solution of claim 5, wherein said organic co-solvent is a ketone solvent.

10. The solution of claim 9, wherein said ketone solvent is acetone.

11. The solution of claim 9, wherein said ketone solvent is methyl ethyl ketone (MEK).

12. The solution of claim 1, further comprising a functional amount of antiseptic agent.

13. The solution of claim 1, further comprising a functional amount of a colorant.

14. A fluid skin covering, comprising
a solution comprising polyether polyurethane (PEPU) resin dissolved in a solvent system comprising 2-methyloxolane (2-MeOX) solvent and at least one co-solvent applied on the skin of a human or other animal, wherein said solution dries to a protective elastic film adhered to said human or other animal skin.

15. A skin protection kit, comprising
a selected volume of a solution comprising polyether polyurethane (PEPU) resin dissolved in a solvent system comprising 2-methyloxolane (2-MeOX) solvent and at least one co-solvent, wherein upon application of said solution to human or other animal skin, said solution dries to a protective elastic film adhered to said human or other animal skin; and
an applicator suitable for applying said solution to said skin.

16. A process for selecting a skin covering solution for a particular application or set of animal skin covering applications, comprising
varying the amount of adhesive agent in a skin covering solution to optimize adhesion of the film formed by said solution on said skin in said application, wherein said solution comprises polyether polyurethane (PEPU) resin dissolved in a mixed solvent comprising 2-methyloxolane (2-MeOX) solvent and at least one co-solvent, and a functional amount of said adhesive agent.

17. A process for protecting human and other animal skins comprising
applying a solution comprising polyether polyurethane (PEPU) resin dissolved in a solvent system comprising 2-methyloxolane (2-MeOX) solvent and at least one co-solvent to said skin.

18. The process of claim 17, wherein said solution further comprises an adhesive agent.

19. The process of claim 17, wherein said solution is applied to the skin using a method selected from the group consisting of dipping, spraying, spreading, brushing and wiping.

20. A method for making a skin covering solution, comprising
dispersing polyether polyurethane and benzoin gum in a mixed solvent comprising 2-methyloxolane (2-MeOX) and a co-solvent that is at least as volatile as 2-MeOX and is acceptable for human skin contact.
